(19) United States
(12) Patent Application Publication
(10) Pub. No.: US 2018/0201989 A1
(43) Pub. Date: Jul. 19, 2018

(54) OXIDANT AND USE THEREOF

(71) Applicant: The University of Tokyo, Tokyo (JP)

(72) Inventor: Seketsu FUKUZAWA, Tokyo (JP)

(73) Assignee: The University of Tokyo, Tokyo (JP)

(21) Appl. No.: 15/743,604

(22) PCT Filed: Jul. 26, 2016
(86) PCT No.: PCT/JP2016/071832

§ 371 Date: Jan. 10, 2018

(30) Foreign Application Priority Data
Jul. 30, 2015 (JP) 2015-150846

(51) Int. Cl. C12Q 1/6858

(52) U.S. Cl. CPC C12Q 1/6858

(57) ABSTRACT

Provided is an oxidant including a hypervalent iodine compound which has iodobenzene micelle-incarcerated by a surfactant as a basic structure and is hydrophobic.

### TECHNICAL FIELD

The present invention relates to an oxidant and use thereof.

Priority is claimed on Japanese Patent Application No. 2015-150846, filed on Jul. 30, 2015, the content of which is incorporated herein by reference.

### BACKGROUND ART

The method for oxidizing a hydroxy group is one of elementary reactions of organic chemistry and many oxidation reagents have been developed thus far. Especially, with respect to the method for quantitatively oxidizing a hydroxy group in an organic solvent, transition metal oxidizing reagents, dimethyl sulfoxide (DMSO), hypervalent iodine reagents such as periodinane compounds, and the like have been developed. However, in an aqueous solvent, strong acid or alkaline conditions are required, and it tends to be difficult to control reactions due to side reactions caused by a hydroxy radical species or the like generated from water molecules. In addition, a protein or DNA, which is a water-soluble biopolymer, has an electron-rich functional group, thus resulting in a macromolecular structure which in turn leads to steric hindrance or the like around the reactive site and therefore belongs to the most difficult class in terms of a substrate. Oxidation of hydroxy groups of a macromolecule in which a wide variety of functional groups are mixed in an aqueous solvent under mild conditions leads to the opening of the way to many possibilities, such as labeling and functionalization, which have never been possible before. For example, hydroxymethylcytosine, which has been attracting attention in the epigenome field, can be converted into uracil by an oxidative or reductive deamination reaction, so if it is possible to develop a mild and quantitative oxidation reaction, the positional information of hydroxymethylcytosine can be obtained at the single base resolution, which can therefore be expected to make a great contribution to stem cell and regenerative medicine research.

PTL 1 discloses a method for identifying a modified cytosine residue, such as 5-hydroxymethylcytosine (5hmC), in a sample nucleotide sequence, using a perruthenate oxidant. The perruthenate oxidant is capable of oxidizing a primary alcohol into an aldehyde and oxidizing a secondary alcohol into a ketone, under weakly basic and mild conditions at room temperature.

In addition, there is an oxidation method using a nitroxy radical called TEMPO, as a mild oxidation method other than use of a transition metal oxidant. Especially, AZADOL (registered trademark) (2-hydroxy-2-azaadamantane) developed by Iwabuchi et al. at Graduate School of Pharmaceutical Sciences, Tohoku University is N-hydroxy which is more highly active than TEMPO (see, for example, PTL 2). AZADOL (registered trademark) has advantages of a higher stability than AZADO (2-azaadamantane-N-oxyl) which is a nitroxy radical and a higher solubility in water than AZADO.

Generally, sodium hypochlorite (NaOCl) or iodobenzene diacetate ((Diacetoxyiodo)benzene or Bis(acetoxy)iodobenzene; BAIB) is used as a co-oxidant. In an aqueous solvent, AZADOL (registered trademark) is oxidized by NaOCl or BAIB to become an oxoammonium salt which is an active species, whereby it is possible to specifically oxidize a hydroxy group of a target water-soluble biopolymer. NaOCl is hydrophilic and BAIB is hydrophobic, and physical properties are conflicting therebetween. In addition, there is a reported case where a hydroxy group of cellulose is oxidized in a homogeneous system using NaOCl as a co-oxidant (see, for example, NPL 1 and 2).

### CITATION LIST

### Patent Literature

[PTL 1] Published Japanese Translation No. 2014-521329 of the PCT International Publication

[PTL 2] PCT International Publication No. WO 2009/145323

### Non-Patent Literature

[NPL 1] Tsuguyuki Saito et al., “Homogeneous Suspensions of Individualized Microfibrils from TEMPO-Catalyzed Oxidation of Native Cellulose”, Biomacromolecules, Volume 7, Number 6, pp. 1687 to 1690, June 2006.
[NPL 2] Tsuguyuki Saito et al., “Cellulose Nanofibers Prepared by TEMPO-Mediated Oxidation of Native Cellulose”, Biomacromolecules, Volume 8, Number 8, pp. 2485 to 2491, Apr. 10, 2007.

### SUMMARY OF INVENTION

### Technical Problem

The perruthenate oxidant described in PTL 1 acts as a trivalent oxidant and is reduced into a ruthenium dioxide. In addition, the perruthenate oxidant has an autocatalytic activity of a reaction, and such an autocatalytic activity is believed to be due to adsorption and then activation of the perruthenate oxidant on the surfaces of insoluble ruthenium dioxide particles generated by the reaction. However, if water is present, water is more preferentially adsorbed onto the surfaces of ruthenium dioxide particles than the perruthenate oxidant, which could possibly present a problem of interfering with an autocatalytic activity. Therefore, a desiccant is needed to adsorb and remove water and there is a difficulty in performing an oxidation reaction in an aqueous solvent that reflects an in vivo environment.

Further, with respect to an oxidation reaction using AZADOL (registered trademark) and NaOCl or BAIB, in the case where NaOCl is used as a co-oxidant, substrates susceptible to an oxidation reaction are decomposed due to radicals generated from NaOCl, which thus results in limitation of substrates to be used. In addition, in the case where BAIB is used as a co-oxidant, it is highly hydrophobic and there is a difficulty in performing an oxidation reaction in an aqueous solvent that reflects an in vivo environment.

The present invention has been made in view of the above-mentioned circumstances and provides a method for selectively oxidizing a hydroxy group in a water-soluble biopolymer in an aqueous solvent that reflects an in vivo environment, under mild conditions.

### Solution to Problem

The present invention includes the following aspects.

[1] An oxidant including a hypervalent iodine compound which has iodobenzene micelle-incarcerated by a surfactant as a basic structure and is hydrophobic.

[2] The oxidant according to [1], in which the surfactant is an anionic surfactant.

[3] The oxidant according to [1] or [2], in which the hypervalent iodine compound is a Dess-Martin reagent or a 2-iodoxybenzoic acid.

[4] The oxidant according to any one of [1] to [3], in which the particle size of the micelle is 50 nm or less.

[5] A method for producing a hypervalent iodine compound micelle-incarcerated by a surfactant, including:

(a) a step of mixing the hypervalent iodine compound, acetonitrile, a surfactant, and a solvent, and shaking and stirring the reaction mixture liquid at a temperature of 4° C. or higher and lower than 25° C. until the color of the liquid changes from transparent to pale yellow; and

(b) a step of further shaking and stirring the reaction mixture liquid at a temperature of 25° C. or higher and 30° C. or lower after the step (a),

in which the hypervalent iodine compound has iodobenzene as a basic structure and is hydrophobic.

[6] The production method according to [5], in which the concentration of the hypervalent iodine compound is 20 mg/mL or more and 30 mg/mL or less.

[7] An oxidation reagent including the oxidant according to any one of [1] to [4], a catalyst which is a compound having an alicyclic heterocyclic ring containing one nitrogen atom as a heteroatom and having a hydroxy group bonded to the nitrogen atom, and a solvent having a pH of 6.0 or more and less than 7.0.

[8] A method for selectively oxidizing a hydroxy group of a target water-soluble biopolymer using the oxidation reagent according to [7].

[9] A kit for selectively oxidizing a hydroxy group of a target water-soluble biopolymer, including a hypervalent iodine compound which has iodobenzene as a basic structure and is hydrophobic, a surfactant, acetonitrile, a catalyst which is a compound having an alicyclic heterocyclic ring containing one nitrogen atom as a heteroatom and having a hydroxy group bonded to the nitrogen atom, and a solvent having a pH of 6.0 or more and less than 7.0.

[10] A kit for detecting hydroxymethylcytosine in a double-stranded polynucleotide, including a hypervalent iodine compound which has iodobenzene as a basic structure and is hydrophobic, a surfactant, acetonitrile, a catalyst which is a compound having an alicyclic heterocyclic ring containing one nitrogen atom as a heteroatom and having a hydroxy group bonded to the nitrogen atom, a solvent having a pH of 6.0 or more and less than 7.0, bisulfite, and a forward and reverse primer set for amplifying a region containing hydroxymethylcytosine in the double-stranded polynucleotide.

[11] A method for detecting hydroxymethylcytosine in a double-stranded polynucleotide, including:

(A) a step of mixing a sample containing a double-stranded polynucleotide having hydroxymethylcytosine, the oxidant according to any one of [1] to [4], a catalyst which is a compound having an alicyclic heterocyclic ring containing one nitrogen atom as a heteroatom and having a hydroxy group bonded to the nitrogen atom, and a solvent having a pH of 6.0 or more and less than 7.0 and oxidizing hydroxymethylcytosine to convert into formylcytosine;

(B) a step of treating a sample containing a double-stranded polynucleotide having the converted formylcytosine with bisulfite to convert formylcytosine into uracil;

(C) a step of adding forward and reverse primers for amplifying a region containing hydroxymethylcytosine in the double-stranded polynucleotide to a double-stranded polynucleotide having the converted uracil to amplify the polynucleotide; and

(D) a step of comparing with a sample containing a double-stranded polynucleotide subjected to steps (A) to (C) without adding an oxidant as a control to detect hydroxymethylcytosine in a double-stranded polynucleotide.

[12] An oxidant containing iodobenzene diacetate micelle-incarcerated by a surfactant.

[13] A method for producing iodobenzene diacetate micelle-incarcerated by a surfactant, including:

(a) a step of mixing iodobenzene diacetate, acetonitrile, a surfactant, and a solvent, and shaking and stirring the reaction mixture liquid at a temperature of 4° C. or higher and lower than 25° C. until the color of the liquid changes from transparent to pale yellow; and

(b) a step of further shaking and stirring the reaction mixture liquid at a temperature of 25° C. or higher and 30° C. or lower after the step (a).

[14] An oxidation reagent including the oxidant according to [12], a catalyst which is a compound having an alicyclic heterocyclic ring containing one nitrogen atom as a heteroatom and having a hydroxy group bonded to the nitrogen atom, and a solvent having a pH of 6.0 or more and less than 7.0.

[15] A method for selectively oxidizing a hydroxy group of a target water-soluble biopolymer using the oxidation reagent according to [14].

[16] A kit for selectively oxidizing a hydroxy group of a target water-soluble biopolymer, including iodobenzene diacetate, a surfactant, acetonitrile, a catalyst which is a compound having an alicyclic heterocyclic ring containing one nitrogen atom as a heteroatom and having a hydroxy group bonded to the nitrogen atom, and a solvent having a pH of 6.0 or more and less than 7.0.

[17] A kit for detecting hydroxymethylcytosine in a double-stranded polynucleotide, including iodobenzene diacetate, a surfactant, acetonitrile, a catalyst which is a compound having an alicyclic heterocyclic ring containing one nitrogen atom as a heteroatom and having a hydroxy group bonded to the nitrogen atom, a solvent having a pH of 6.0 or more and less than 7.0, bisulfite, and a forward and reverse primer set for amplifying a region containing hydroxymethylcytosine in the double-stranded polynucleotide.

[18] A method for detecting hydroxymethylcytosine in a double-stranded polynucleotide, including:

(A) a step of mixing a sample containing a double-stranded polynucleotide having hydroxymethylcytosine, the oxidant according to [12], a catalyst which is a compound having an alicyclic heterocyclic ring containing one nitrogen atom as a heteroatom and having a hydroxy group bonded to the nitrogen atom, and a solvent having a pH of 6.0 or more and less than 7.0 and oxidizing hydroxymethylcytosine to convert into formylcytosine;

(B) a step of treating a sample containing a double-stranded polynucleotide having the converted formylcytosine with bisulfite to convert formylcytosine into uracil;

(C) a step of adding forward and reverse primers for amplifying a region containing hydroxymethylcytosine in the double-stranded polynucleotide to a double-stranded polynucleotide having the converted uracil to amplify the polynucleotide; and

(D) a step of comparing with a sample containing a double-stranded polynucleotide subjected to steps (A) to (C) without adding an oxidant as a control to detect hydroxymethylcytosine in a double-stranded polynucleotide.

### Advantageous Effects of Invention

According to the present invention, it is possible to selectively oxidize a hydroxy group in a water-soluble biopolymer in an aqueous solvent that reflects an in vivo environment, under mild conditions. Further, according to the present invention, it is possible to easily detect hydroxymethylcytosine in a double-stranded polynucleotide.

### BRIEF DESCRIPTION OF DRAWINGS

FIG. 1 is a schematic diagram showing an oxidation reaction of hydroxymethylcytosine using an oxidant containing micelle-incarcerated BAIB and AZADOL (registered trademark) in the present embodiment.

FIG. 2 is a diagram comparing changes in hydroxymethylcytosine and methylcytosine in a double-stranded polynucleotide before and after subjecting to an oxidation reaction and a bisulfite treatment in the present embodiment.

FIG. 3 is a diagram comparing changes in hydroxymethylcytosine and methylcytosine in a double-stranded polynucleotide before and after subjecting to an oxidation reaction and a bisulfite treatment in the present embodiment.

FIG. 4 is a graph showing the results of LC-ESI-MS measurement of substrate DNA in Example 1.

FIG. 5A is a schematic diagram showing the position of hydroxymethylcytosine and methylcytosine in substrate DNA, and the conversion of cytosine and hydroxymethylcytosine into uracil in substrate DNA by means of an oxidation reaction and a bisulfite treatment in Example 2.

FIG. 5B is a schematic diagram showing changes in the conversion rate of hydroxymethylcytosine into uracil depending on a difference of an oxidation reaction time of substrate DNA in Example 2.

FIG. 6 is a graph showing changes in the percentage of methylated (or hydroxymethylated) cytosine depending on a difference of an oxidation reaction time of substrate DNA in Example 2.

### DESCRIPTION OF EMBODIMENTS

Hereinafter, embodiments of the present invention will be described in detail with reference to the drawings as necessary.

In the drawings, the same or corresponding parts are denoted by the same reference numerals and redundant description is therefore omitted.

<<Oxidant>>

In one embodiment, the present invention provides an oxidant containing a hypervalent iodine compound which has iodobenzene micelle-incarcerated by a surfactant as a basic structure and is hydrophobic.

According to the present embodiment, it is possible to use a hypervalent iodine compound which has iodobenzene as a basic structure and is hydrophobic in an aqueous solvent as an oxidant. Further, by micelle-incarcerating a hypervalent iodine compound which has iodobenzene as a basic structure and is hydrophobic, it is possible to isolate the hypervalent iodine compound from a substrate, thus capable of preventing side reactions such as decomposition of the substrate.

As used herein, the term “surfactant” refers to a substance which acts on an interface (boundary surface of a substance) to result in changes of properties. The surfactant has two parts of a “hydrophilic” moiety which readily dissolves in water and a “lipophilic” moiety which readily dissolves in oil in one molecule in terms of a structure. In the present embodiment, the surfactant to be used is preferably an ionic surfactant and more preferably an anionic surfactant.

As used herein, the term “anionic surfactant” refers to a surfactant in which a hydrophilic group (moiety which structurally dissolves in water) dissociates into negative ions by the surfactant being ionized when dissolved in water. With regard to the structure, the anionic surfactant has a carboxylic acid, sulfonic acid, or phosphoric acid structure as a hydrophilic group, examples thereof include a carboxylic acid type (R—COO-M+), a branched or linear sodium alkylbenzene sulfonate (R—C₆H₄—SO₃⁻Na⁺), a sulfonic acid type (R—SO₃⁻Na⁺), a sulfate ester type (R—O—SO₃⁻Na⁺), and a phosphate ester type (R—O—PO(OH)OM⁺). Specific examples of the anionic surfactant include a sodium cholate hydrate, sodium deoxycholate, sodium glycocholate, sodium taurocholate, sodium taurodeoxycholate, sodium N-lauroyl sarcosinate, lithium dodecyl sulfate, sodium dodecyl sulfate (SDS), and dodecyl phosphocholine (DPC). Among these, SDS or DPC is particularly preferable in terms of being able to produce an inexpensive and compact micelle.

As used herein, the term “oxidation” refers to a chemical reaction in which a target substance, such as a water-soluble biopolymer, loses an electron. Specific examples of the oxidation include a reaction in which an oxygen atom combines with a target substance and a reaction in which a target substance is deprived of a hydrogen atom.

As used herein, the term “oxidant” refers to a compound from which an oxygen atom is to be transferred or a substance capable of acquiring an electron by an oxidation-reduction reaction, and is an agent that oxidizes a hydroxyl group in a target substance, such as a water-soluble biopolymer, into an aldehyde or a ketone.

<Hypervalent Iodine Compound>

Generally, the hypervalent iodine compound is an iodine-containing compound having eight or more electrons in a valence shell.

The hypervalent iodine compound in the present embodiment is a compound represented by the following General Formula (1) (hereinafter, sometimes referred to simply as “compound (1)”) or a compound represented by the following General Formula (2) (hereinafter, sometimes referred to simply as “compound (2)”) each of which has iodobenzene as a basic structure and is hydrophobic.

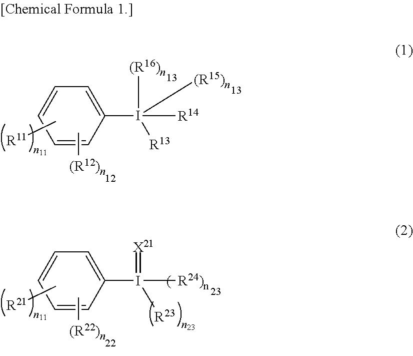

[Groups represented by R¹¹, R¹⁴, R¹⁵, R¹⁶, R²¹, and R²⁴]

In General Formulae (1) and (2), R¹¹, R¹⁴, R¹⁵, R¹⁶, R²¹, and R²⁴ are each independently a hydrogen atom, a halogen atom, a nitro group, a cyano group, a hydroxy group, a mercapto group, an amino group, a formyl group, a carboxyl group, a sulfo group, a C₁₋₁₂ alkyl group, a C₃₋₁₂ cycloalkyl group, a (C₁₋₁₂ alkyl)oxy group (C₁₋₁₂ alkoxy group), a (C₃₋₁₂ cycloalkyl)oxy group, a (C₁₋₁₂ alkyl)thio group, a (C₃₋₁₂ cycloalkyl)thio group, a (C₁₋₁₂ alkyl)amino group, a (C₃₋₁₂ cycloalkyl)amino group, a di(C₁₋₆ alkyl)amino group, a di(C₃₋₆ cycloalkyl)amino group, a C₁₋₁₂ alkylcarbonyl group, a C₃₋₁₂ cycloalkylcarbonyl group, a (C₁₋₁₂ alkyl)oxycarbonyl group, a (C₃₋₁₂ cycloalkyl)oxycarbonyl group, a (C₁₋₁₂ alkyl)thiocarbonyl group, a (C₃₋₁₂ cycloalkyl)thiocarbonyl group, a (C₁₋₁₂ alkyl)aminocarbonyl group, a (C₃₋₁₂ cycloalkyl)aminocarbonyl group, a di(C₁₋₆ alkyl)aminocarbonyl group, a di(C₃₋₆ cycloalkyl)aminocarbonyl group, a (C₁₋₁₂ alkyl)carbonyloxy group, a (C₃₋₁₂ cycloalkyl)carbonyloxy group, a (C₁₋₁₂ alkyl)carbonylthio group, a (C₃₋₁₂ cycloalkyl)carbonylthio group, a (C₁₋₁₂ alkyl)carbonylamino group, a (C₃₋₁₂ cycloalkyl)carbonylamino group, a di(C₁₋₁₂ alkylcarbonyl)amino group, a di(C₃₋₁₂ cycloalkylcarbonyl)amino group, a C₁₋₆ haloalkyl group, a C₃₋₆ halocycloalkyl group, a C₂₋₆ alkenyl group, a C₃₋₆ cycloalkenyl group, a C₂₋₆ haloalkenyl group, a C₃₋₆ halocycloalkenyl group, a C₂₋₆ alkynyl group, a C₂₋₆ haloalkynyl group, a benzyl group which may be substituted by R^{a}, a benzyloxy group which may be substituted by R^{a}, a benzylthio group which may be substituted by R^{a}, a benzylamino group which may be substituted by R^{a}, a dibenzylamino group which may be substituted by R^{a}, a benzylcarbonyl group which may be substituted by R^{a}, a benzyloxycarbonyl group which may be substituted by R^{a}, a benzylthiocarbonyl group which may be substituted by R^{a}, a benzylaminocarbonyl group which may be substituted by R^{a}, a dibenzylaminocarbonyl group which may be substituted by R^{a}, a benzylcarbonyloxy group which may be substituted by R^{a}, a benzylcarbonylthio group which may be substituted by R^{a}, a benzylcarbonylamino group which may be substituted by R^{a}, a di(benzylcarbonyl)amino group which may be substituted by R^{a}, an aryl group which may be substituted by R^{a}, an aryloxy group which may be substituted by R^{a}, an arylthio group which may be substituted by R^{a}, an arylamino group which may be substituted by R^{a}, a diarylamino group which may be substituted by R^{a}, an arylcarbonyl group which may be substituted by R^{a}, an aryloxycarbonyl group which may be substituted by R^{a}, an arylthiocarbonyl group which may be substituted by R^{a}, an arylaminocarbonyl group which may be substituted by R^{a}, a diarylaminocarbonyl group which may be substituted by R^{a}, an arylcarbonyloxy group which may be substituted by R^{a}, an arylcarbonylthio group which may be substituted by R^{a}, an arylcarbonylamino group which may be substituted by R^{a}, a di(arylcarbonyl)amino group which may be substituted by R^{a}, or a benzenesulfonyloxy group which may be substituted by R^{a}. R¹¹, R¹⁴, R¹⁵, and R¹⁶ may be the same or different from one another.

Examples of the halogen atom include a fluorine atom, a chlorine atom, a bromine atom, and an iodine atom. In addition, the term “halo” as used herein also refers to such a halogen atom.

The term “Cₐ-C_{b} alkyl” as used herein refers to a linear or branched hydrocarbon group having a to b number of carbon atoms.

Specific examples of the Cₐ-C_{b} alkyl include a methyl group, an ethyl group, an n-propyl group, an i-propyl group, an n-butyl group, an i-butyl group, an s-butyl group, a t-butyl group, an n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, an n-hexyl group, a 1-methylpentyl group, a 2-methylpentyl group, a 1,1-dimethylbutyl group, a 1,3-dimethylbutyl group, a heptyl group, an octyl group, a nonyl group, a decyl group, an undecyl group, and a dodecyl group, each of which is selected in the range of the specified number of carbon atoms.

The term “Cₐ-C_{b} haloalkyl” as used herein refers to a linear or branched hydrocarbon group having a to b number of carbon atoms in which a hydrogen atom bonded to a carbon atom may be optionally substituted by a halogen atom. In the case of being substituted by two or more halogen atoms, those halogen atoms may be the same or different from one another.

Specific examples of the Cₐ-C_{b} haloalkyl include a fluoromethyl group, a chloromethyl group, a bromomethyl group, an iodomethyl group, a difluoromethyl group, a chlorofluoromethyl group, a dichloromethyl group, a bromofluoromethyl group, a trifluoromethyl group, a chlorodifluoromethyl group, a dichlorofluoromethyl group, a trichloromethyl group, a bromodifluoromethyl group, a bromochlorofluoromethyl group, a dibromofluoromethyl group, a 2-fluoroethyl group, a 2-chloroethyl group, a 2-bromoethyl group, a 2,2-difluoroethyl group, a 2-chloro-2-fluoroethyl group, a 2,2-dichloroethyl group, a 2-bromo-2-fluoroethyl group, a 2,2,2-trifluoroethyl group, a 2-chloro-2,2-difluoroethyl group, a 2,2-dichloro-2-fluoroethyl group, a 2,2,2-trichloroethyl group, a 2-bromo-2,2-difluoroethyl group, a 2-bromo-2-chloro-2-fluoroethyl group, a 2-bromo-2,2-dichloroethyl group, a 1,1,2,2-tetrafluoroethyl group, a pentafluoroethyl group, a 1-chloro-1,2,2,2-tetrafluoroethyl group, a 2-chloro-1,1,2,2-tetrafluoroethyl group, a 1,2-dichloro-1,2,2-trifluoroethyl group, a 2-bromo-1,1,2,2-tetrafluoroethyl group, a 2-fluoropropyl group, a 2-chloropropyl group, a 2-bromopropyl group, a 2-chloro-2-fluoropropyl group, a 2,3-dichloropropyl group, a 2-bromo-3-fluoropropyl group, a 3-bromo-2-chloropropyl group, a 2,3-dibromopropyl group, a 3,3,3-trifluoropropyl group, a 3-bromo-3,3-difluoropropyl group, a 2,2,3,3-tetrafluoropropyl group, a 2-chloro-3,3,3-trifluoropropyl group, a 2,2,3,3,3-pentafluoropropyl group, a 1,1,2,3,3,3-hexafluoropropyl group, a heptafluoropropyl group, a 2,3-dichloro-1,1,2,3,3-pentafluoropropyl group, a 2-fluoro-1-methylethyl group, a 2-chloro-1-methylethyl group, a 2-bromo-1-methylethyl group, a 2,2,2-trifluoro-1-(trifluoromethyl)ethyl group, a 1,2,2,2-tetrafluoro-1-(trifluoromethyl)ethyl group, a 2,2,3,3,4,4-hexafluorobutyl group, a 2,2,3,4,4,4-hexafluorobutyl group, a 2,2,3,3,4,4,4-heptafluorobutyl group, a 1,1,2,2,3,3,4,4-octafluorobutyl group, a nonafluorobutyl group, a 4-chloro-1,1,2,2,3,3,4,4-octafluorobutyl group, a 2-fluoro-2-methylpropyl group, a 2-chloro-1,1-dimethylethyl group, a 2-bromo-1,1-dimethylethyl group, a 5-chloro-2,2,3,4,4,5,5-heptafluoropentyl group, and a tridecafluorohexyl group, each of which is selected in the range of the specified number of carbon atoms.

The term “Cₐ-C_{b} cycloalkyl” as used herein refers to a cyclic hydrocarbon group having a to b number of carbon atoms and is capable of forming a 3- to 6-membered monocyclic or polycyclic ring structure. In addition, each ring may be optionally substituted by an alkyl group in the range of the specified number of carbon atoms.

Specific examples of the Cₐ-C_{b} cycloalkyl include a cyclopropyl group, a 1-methylcyclopropyl group, a 2-methylcyclopropyl group, a 2,2-dimethylcyclopropyl group, a 2,2,3,3-tetramethylcyclopropyl group, a cyclobutyl group, a cyclopentyl group, a 2-methylcyclopentyl group, a 3-methylcyclopentyl group, a cyclohexyl group, a 2-methylcyclohexyl group, a 3-methylcyclohexyl group, a 4-methylcyclohexyl group, and a bicyclo[2.2.1]heptan-2-yl group, each of which is selected in the range of the specified number of carbon atoms.

The term “Cₐ-C_{b} halocycloalkyl” as used herein refers to a cyclic hydrocarbon group having a to b number of carbon atoms in which a hydrogen atom bonded to a carbon atom may be optionally substituted by a halogen atom, and is capable of forming a 3- to 6-membered monocyclic or polycyclic ring structure. In addition, each ring may be optionally substituted by an alkyl group in the range of the specified number of carbon atoms, the substitution by a halogen atom may be of a ring structure moiety, a side chain moiety or both thereof, and in the case of being substituted by two or more halogen atoms, those halogen atoms may be the same or different from one another.

Specific examples of the Cₐ-C_{b} halocycloalkyl include a 2,2-difluorocyclopropyl group, a 2,2-dichlorocyclopropyl group, a 2,2-dibromocyclopropyl group, a 2,2-difluoro-1-methylcyclopropyl group, a 2,2-dichloro-1-methylcyclopropyl group, a 2,2-dibromo-1-methylcyclopropyl group, a 2,2,3,3-tetrafluorocyclobutyl group, a 2-(trifluoromethyl)cyclohexyl group, a 3-(trifluoromethyl)cyclohexyl group, and a 4-(trifluoromethyl)cyclohexyl group, each of which is selected in the range of the specified number of carbon atoms.

The term “Cₐ-C_{b} alkenyl” as used herein refers to a linear or branched unsaturated hydrocarbon group having a to b number of carbon atoms and having one or two or more double bonds in the molecule.

Specific examples of the Cₐ-C_{b} alkenyl include a vinyl group, a 1-propenyl group, a 2-propenyl group, a 1-methylethenyl group, a 2-butenyl group, a 1-methyl-2-propenyl group, a 2-methyl-2-propenyl group, a 2-pentenyl group, a 2-methyl-2-butenyl group, a 3-methyl-2-butenyl group, a 2-ethyl-2-propenyl group, a 1,1-dimethyl-2-propenyl group, a 2-hexenyl group, a 2-methyl-2-pentenyl group, a 2,4-dimethyl-2,6-heptadienyl group, and a 3,7-dimethyl-2,6-octadienyl group, each of which is selected in the range of the specified number of carbon atoms.

The term “Cₐ-C_{b} haloalkenyl” as used herein refers to a linear or branched unsaturated hydrocarbon group having a to b number of carbon atoms and having one or two or more double bonds in the molecule, in which a hydrogen atom bonded to a carbon atom may be optionally substituted by a halogen atom. In the case of being substituted by two or more halogen atoms, those halogen atoms may be the same or different from one another.

Specific examples of the Cₐ-C_{b} haloalkenyl include a 2,2-dichlorovinyl group, a 2-fluoro-2-propenyl group, a 2-chloro-2-propenyl group, a 3-chloro-2-propenyl group, a 2-bromo-2-propenyl group, a 3-bromo-2-propenyl group, a 3,3-difluoro-2-propenyl group, a 2,3-dichloro-2-propenyl group, a 3,3-dichloro-2-propenyl group, a group, a 2,3-dichloro-2-propenyl group, a 3,3-dichloro-2-propenyl group, a 2,3-dibromo-2-propenyl group, a 2,3,3-trifluoro-2-propenyl group, a 2,3,3-trichloro-2-propenyl group, a 1-(trifluoromethyl)ethenyl group, a 3-chloro-2-butenyl group, a 3-bromo-2-butenyl group, a 4,4-difluoro-3-butenyl group, a 3,4,4-trifluoro-3-butenyl group, a 3-chloro-4,4,4-trifluoro-2-butenyl group, and a 3-bromo-2-methyl-2-propenyl group, each of which is selected in the range of the specified number of carbon atoms.

The term “Cₐ-C_{b} alkynyl” as used herein refers to a linear or branched unsaturated hydrocarbon group having a to b number of carbon atoms and having one or two or more triple bonds in the molecule.

Specific examples of the Cₐ-C_{b} alkynyl include an ethynyl group, a 1-propynyl group, a 2-propynyl group, a 2-butynyl group, a 1-methyl-2-propynyl group, a 2-pentynyl group, a 1-methyl-2-butynyl group, a 1,1-dimethyl-2-propynyl group, and a 2-hexynyl group, each of which is selected in the range of the specified number of carbon atoms.

The term “Cₐ-C_{b} haloalkynyl” as used herein refers to a linear or branched unsaturated hydrocarbon group having a to b number of carbon atoms and having one or two or more triple bonds in the molecule, in which a hydrogen atom bonded to a carbon atom may be optionally substituted by a halogen atom. In the case of being substituted by two or more halogen atoms, those halogen atoms may be the same or different from one another.

Specific examples of the Cₐ-C_{b} haloalkynyl include a 2-chloroethynyl group, a 2-bromoethynyl group, a 2-iodoethynyl group, a 3-chloro-2-propynyl group, a 3-bromo-2-propynyl group, and a 3-iodo-2-propynyl group, each of which is selected in the range of the specified number of carbon atoms.

(Group represented by R^{a})

R^{a} is a halogen atom, a C₁₋₆ alkyl group, a C₁₋₆ haloalkyl group, a C₃₋₆ cycloalkyl group, a C₁₋₆ alkoxy group, a C₁₋₆ alkoxy C₁₋₆ alkyl group, a C₁₋₆ alkylsulfenyl C₁₋₆ alkyl group, a C₁₋₆ haloalkoxy group, a C₁₋₆ alkylsulfenyl group, a C₁₋₆ alkylsulfinyl group, a C₁₋₆ alkylsulfonyl group, a C₁₋₆ haloalkylsulfenyl group, a C₁₋₆ haloalkylsulfinyl group, a C₁₋₆ haloalkylsulfonyl group, a C₂₋₆ alkenyl group, a C₂₋₆ haloalkenyl group, a C₂₋₆ alkenyloxy group, a C₂₋₆ haloalkenyloxy group, a C₂₋₆ alkenylsulfenyl group, a C₂₋₆ alkenylsulfinyl group, a C₂₋₆ alkenylsulfonyl group, a C₂₋₆ haloalkenylsulfenyl group, a C₂₋₆ haloalkenylsulfinyl group, a C₂₋₆ haloalkenylsulfonyl group, a C₂₋₆ alkynyl group, a C₂₋₆ haloalkynyl group, a C₂₋₆ alkynyloxy group, a C₂₋₆ haloalkynyloxy group, a C₂₋₆ alkynylsulfenyl group, a C₂₋₆ alkynylsulfinyl group, a C₂₋₆ alkynylsulfonyl group, a C₂₋₆ haloalkynylsulfenyl group, a C₂₋₆ haloalkynylsulfinyl group, a C₂₋₆ haloalkynylsulfonyl group, —NO₂, —CN, a formyl group, —OH, —SH, —NH₂, —SCN, a C₁₋₆ alkoxycarbonyl group, a C₁₋₆ alkylcarbonyl group, a C₁₋₆ haloalkylcarbonyl group, a C₁₋₆ alkylcarbonyloxy group, a phenyl group, a C₁₋₆ alkylamino group, or a di-C₁₋₆ alkylamino group. The number of R^{a} to be substituted is 1 to 5, and in the case where the number of R^{a} is 2 or more, the respective substituents may be the same or different.

Examples of the aryl group which may be substituted by R^{a} include, but are not limited to, a phenyl group, an o-methylphenyl group, an m-methylphenyl group, a p-methylphenyl group, an o-chlorophenyl group, an m-chlorophenyl group, a p-chlorophenyl group, an o-fluorophenyl group, a p-fluorophenyl group, an o-methoxyphenyl group, a p-methoxyphenyl group, a p-nitrophenyl group, a p-cyanophenyl group, an a-naphthyl group, a β-naphthyl group, an o-biphenylyl group, an m-biphenylyl group, a p-biphenylyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 2-thienyl group, a 3-thienyl group, a 2-furyl group, a 3-furyl group, a 2-pyranyl group, a 3-pyranyl group, a 4-pyranyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 4-isobenzofuranyl group, a 5-isobenzofuranyl group, a 2-benzothienyl group, a 3-benzothienyl group, a 4-benzothienyl group, a 5-benzothienyl group, a 6-benzothienyl group, a 7-benzothienyl group, a 1-isobenzothienyl group, a 4-isobenzothienyl group, a 5-isobenzothienyl group, a 2-chromenyl group, a 3-chromenyl group, a 4-chromenyl group, a 5-chromenyl group, a 6-chromenyl group, a 7-chromenyl group, a 8-chromenyl group, a 1-pyrrolyl group, a 2-pyrrolyl group, a 3-pyrrolyl group, a 1-imidazolyl group, a 2-imidazolyl group, a 4-imidazolyl group, a 1-pyrazolyl group, a 3-pyrazolyl group, a 4-pyrazolyl group, a 2-thiazolyl group, a 4-thiazolyl group, a 5-thiazolyl group, a 3-isothiazolyl group, a 4-isothiazolyl group, a 5-isothiazolyl group, a 2-oxazolyl group, a 4-oxazolyl group, a 5-oxazolyl group, a 3-isoxazolyl group, a 4-isoxazolyl group, a 5-isoxazolyl group, a 2-pyridyl group, a 3-pyridyl group, a 4-pyridyl group, a 2-pyrazinyl group, a 2-pyrimidinyl group, a 4-pyrimidinyl group, a 5-pyrimidinyl group, a 3-pyridazinyl group, a 4-pyridazinyl group, a 1-indolizinyl group, a 2-indolizinyl group, a 3-indolizinyl group, a 5-indolizinyl group, a 6-indolizinyl group, a 7-indolizinyl group, a 8-indolizinyl group, a 1-isoindolyl group, a 4-isoindolyl group, a 5-isoindolyl group, a 1-indolyl group, a 2-indolyl group, a 3-indolyl group, a 4-indolyl group, a 5-indolyl group, a 6-indolyl group, a 7-indolyl group, a 1-indazolyl group, a 2-indazolyl group, a 3-indazolyl group, a 4-indazolyl group, a 5-indazolyl group, a 6-indazolyl group, a 7-indazolyl group, a 1-prenyl group, a 2-prenyl group, a 3-prenyl group, a 6-prenyl group, a 7-prenyl group, a 8-prenyl group, a 2-quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 1-phthalazinyl group, a 5-phthalazinyl group, a 6-phthalazinyl group, a 2-naphthyridinyl group, a 3-naphthyridinyl group, a 4-naphthyridinyl group, a 2-quinoxalinyl group, a 5-quinoxalinyl group, a 6-quinoxalinyl group, a 2-quinazolinyl group, a 4-quinazolinyl group, a 5-quinazolinyl group, a 6-quinazolinyl group, a 7-quinazolinyl group, a 8-quinazolinyl group, a 3-cinnolinyl group, a 4-cinnolinyl group, a 5-cinnolinyl group, a 6-cinnolinyl group, a 7-cinnolinyl group, a 8-cinnolinyl group, a 2-ptenidinyl group, 4-ptenidinyl group, 6-ptenidinyl group, 7-ptenidinyl group, and a 3-furazanyl group.

Examples of the benzyl group which may be substituted by R^{a} include, but are not limited to, a benzyl group, an o-methylbenzyl group, an m-methylbenzyl group, a p-methylbenzyl group, an o-chlorobenzyl group, an m-chlorobenzyl group, a p-chlorobenzyl group, an o-fluorobenzyl group, a p-fluorobenzyl group, an o-methoxybenzyl group, a p-methoxybenzyl group, a p-nitrobenzyl group, and a p-cyanobenzyl group.

Examples of the benzenesulfonyloxy group which may be substituted by R^{a} include, but are not limited to, a benzenesulfonyloxy group, an o-toluenesulfonyloxy group, an m-toluenesulfonyloxy group, a p-toluenesulfonyl group, an o-chlorobenzenesulfonyloxy group, an m-chlorobenzenesulfonyloxy group, a p-chlorobenzenesulfonyloxy group, an o-fluorobenzenesulfonyloxy group, a p-fluorobenzenesulfonyloxy group, an o-methoxybenzenesulfonyloxy group, a p-methoxybenzenesulfonyloxy group, a p-nitrobenzenesulfonyloxy group, and a p-cyanobenzenesulfonyloxy group.

Among them, R¹¹ and R²¹ are preferably a hydrogen atom.

Among them, R¹⁴, R¹⁵, R¹⁶, and R²⁴ are preferably a fluorine atom, a hydroxy group, a trifluoromethyl group, a methylcarbonyloxy group, a trifluoromethylcarbonyloxy group, a p-toluenesulfonyloxy group, or a phenyl group.

R¹⁴, R¹⁵, and R¹⁶ are preferably identical from the viewpoint of easy synthesis.

[Groups represented by R¹², R¹³, R²², and R²³]

In General Formulae (1) and (2), R¹², R¹³, R²², and R²³ each independently have the same definition as R¹¹, R¹⁴, R¹⁵, and R¹⁶. In the present embodiment, R¹² and R¹³, or R²² and R²³ may be bonded to each other to form —O—, —S—, —NH—, —CO—, —COO—, —CONH—, a C₁₋₁₀ alkylene group, or a combination thereof.

The term “Cₐ-C_{b} alkylene” as used herein refers to a linear or branched unsaturated hydrocarbon group having a to b number of carbon atoms and having one or two or more double bonds in the molecule.

Specific examples of the Cₐ-C_{b} alkylene include linear alkylene groups such as a methylene group, an ethylene group, an n-propylene group, an n-butylene group, an n-pentylene group, an n-hexylene group, an n-heptylene group, an n-octylene group, an n-nonylene group, and an n-decylene group, and branched alkylene groups such as a dimethylmethylene group, a 2-methylpropylene group, a 2-methylhexylene group, and a tetramethylethylene group, each of which is selected in the range of the specified number of carbon atoms.

In the case where R¹² and R¹³, or R²² and R²³ are bonded to each other, in particular, it is preferred that R¹² and R¹³, or R²² and R²³ form —COO— or a dimethylmethyleneoxy group.

In the case where R¹² and R¹³, or R²² and R²³ are not bonded to each other, in particular, R¹² and R²² are preferably a hydrogen atom, and R¹³ and R²³ are preferably a fluorine atom, a hydroxy group, a trifluoromethyl group, a methylcarbonyloxy group, a trifluoromethylcarbonyloxy group, a p-toluenesulfonyloxy group, or a phenyl group.

[X²¹]

In General Formula (2), X²¹ is an oxygen atom or a sulfur atom. Among them, X²¹ is preferably an oxygen atom.

[n₁₁, n₁₂, n₁₃, n₂₁, n₂₂, and n₂₃]

In General Formula (1), n₁₁ represents the number of R¹¹ and is an integer of any one of 1 to 5. When n₁₁ is 2 or more, R¹¹'s may be the same or different from one another.

In General Formula (1), n₁₂ represents the number of R¹² and is an integer of any one of 0 to 4. When n₁₂ is 2 or more, R¹²'s may be the same or different from one another.

In General Formula (2), n₂₁ represents the number of R²¹ and is an integer of any one of 1 to 5. When n₂₁ is 2 or more, R²¹'s may be the same or different from one another.

In General Formula (2), n₂₂ represents the number of R²² and is an integer of any one of 0 to 4. When n₂₂ is 2 or more, R²²'s may be the same or different from one another.

The sum of n₁₁ and n₁₂ is 5, and the sum of n₂₁ and n₂₂ is 5.

n₁₃ and n₂₃ are 0 or 1.

Preferred examples of the compound (1) include a compound represented by the following General Formula (1-1) (hereinafter, sometimes referred to simply as “compound (1-1)”) and a compound represented by the following General Formula (1-2) (hereinafter, sometimes referred to simply as “compound (1-2)”).

These compounds are merely examples of the preferred compound (1), and the preferred compound (1) is not limited thereto.

Preferred examples of the compound (2) include a compound represented by the following General Formula (2-1) (hereinafter, sometimes referred to simply as “compound (2-1)”) and a compound represented by the following General Formula (2-2) (hereinafter, sometimes referred to simply as “compound (2-2)”).

These compounds are merely examples of the preferred compound (2), and the preferred compound (2) is not limited thereto.

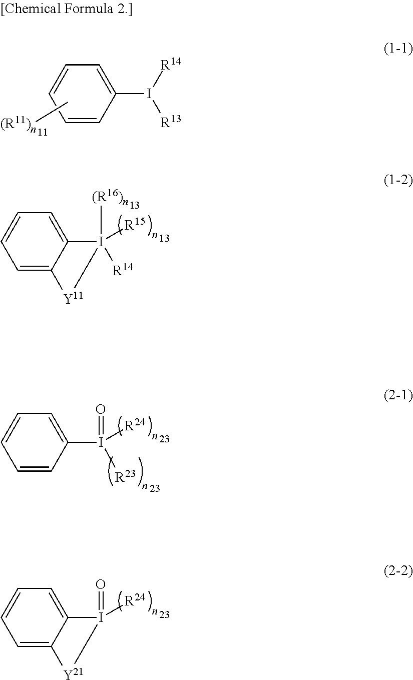

(In the formulae, R¹¹, R¹³, R¹⁴, R¹⁵, R¹⁶, R²³, R²⁴, n₁₁, n₁₃, and n₂₃ are as defined above. Y¹¹ and Y²¹ are —O—, —S—, —NH—, —CO—, —COO—, —CONH—, a C₁₋₁₀ alkylene group, or a combination thereof)

[Y¹¹ and Y²¹]

In General Formulae (1-2) and (2-2), Y¹¹ and Y²¹ are —O—, —S—, —NH—, —CO—, —COO—, —CONH—, a C₁₋₁₀ alkylene group, or a combination thereof. Among them, Y¹¹ and Y²¹ are preferably —COO— or a dimethylmethyleneoxy group.

The compound (1-1) is preferably, for example, a compound in which R¹¹ is a hydrogen atom or a C₁₋₁₂ alkyl group, R¹³ is a halogen atom, a hydroxy group, a trifluoromethyl group, a methylcarbonyloxy group, a trifluoromethylcarbonyloxy group, a p-toluenesulfonyloxy group, or a phenyl group, and R¹⁴ is a halogen atom, a hydroxy group, a trifluoromethyl group, a methylcarbonyloxy group, a trifluoromethylcarbonyloxy group, a p-toluenesulfonyloxy group, or a phenyl group.

The compound (1-1) is more preferably, for example, a compound in which R is a hydrogen atom or a methyl group, R¹³ is a fluorine atom, a hydroxy group, a methylcarbonyloxy group, a trifluoromethylcarbonyloxy group, or a phenyl group, and R¹⁴ is a fluorine atom, a methylcarbonyloxy group, a trifluoromethylcarbonyloxy group, or a p-toluenesulfonyloxy group.

The compound (1-2) is preferably, for example, a compound in which R¹⁴, R¹⁵ and R¹⁶ are a halogen atom, a hydroxy group, a trifluoromethyl group, a methylcarbonyloxy group, a trifluoromethylcarbonyloxy group, a p-toluenesulfonyloxy group, or a phenyl group, and Y¹¹ is —COO— or a dimethylmethyleneoxy group.

The compound (1-2) is more preferably, for example, a compound in which R¹⁴, R¹⁵ and R¹⁶ are a methylcarbonyloxy group, a trifluoromethylcarbonyloxy group, or a phenyl group, and Y¹¹ is —COO—.

The compound (2-1) is preferably, for example, a compound in which R²³ and R²³ are a halogen atom, a hydroxy group, a trifluoromethyl group, a methylcarbonyloxy group, a trifluoromethylcarbonyloxy group, a p-toluenesulfonyloxy group, or a phenyl group.

The compound (2-1) is more preferably, for example, a compound in which R²³ and R²³ are a fluorine atom, a hydroxy group, a methylcarbonyloxy group, a trifluoromethylcarbonyloxy group, or a phenyl group.

The compound (2-2) is preferably, for example, a compound in which R²⁴ is a fluorine atom, a hydroxy group, a trifluoromethyl group, a methylcarbonyloxy group, a trifluoromethylcarbonyloxy group, a p-toluenesulfonyloxy group, or a phenyl group, and Y¹¹ is —COO— or a dimethylmethyleneoxy group.

The compound (2-2) is more preferably, for example, a compound in which R²⁴ is a hydroxy group or a methylcarbonyloxy group, and Y¹¹ is —COO—.

Among the compounds (1), preferred examples of the compound (1-1) include a compound represented by the following General Formula (1-1-1) (hereinafter, sometimes referred to simply as “compound (1-1-1)”), a compound represented by the following General Formula (1-1-2) (hereinafter, sometimes referred to simply as “compound (1-1-2)”), a compound represented by the following General Formula (1-1-3) (hereinafter, sometimes referred to simply as “compound (1-1-3)”), a compound represented by the following General Formula (1-1-4) (hereinafter, sometimes referred to simply as “compound (1-1-4)”), and a compound represented by the following General Formula (1-1-5) (hereinafter, sometimes referred to simply as “compound (1-1-5)”).

Among the compounds (1), preferred examples of the compound (1-2) include a compound represented by the following General Formula (1-2-1) (hereinafter, sometimes referred to simply as “compound (1-2-1)”), and a compound represented by the following General Formula (1-2-2) (hereinafter, sometimes referred to simply as “compound (1-2-2)”).

These compounds are merely examples of the preferred compound (1), and the preferred compound (1) is not limited thereto.

Among the compounds (2), the compound (2-1) is preferably, for example, a compound represented by the following General Formula (2-1-1) (hereinafter, sometimes referred to simply as “compound (2-1-1)”).

Among the compounds (2), the compound (2-2) is preferably, for example, a compound represented by the following General Formula (2-2-1) (hereinafter, sometimes referred to simply as “compound (2-2-1)”).

These compounds are merely examples of the preferred compound (2), and the preferred compound (2) is not limited thereto.

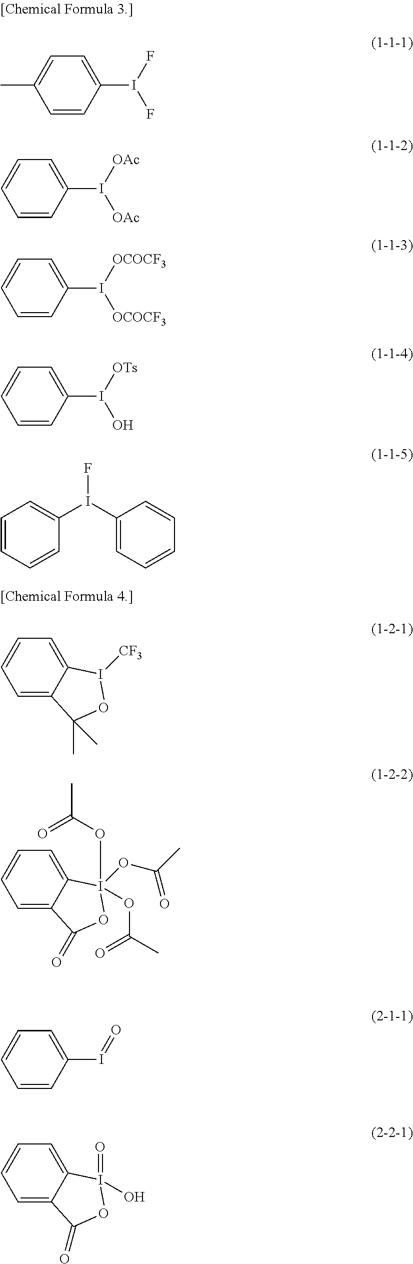

In the formulae, Ac is an acetyl group (methylcarbonyl group), and Ts is a tosyl group (p-toluenesulfonyl group).

The compound (1-1-2) is a compound called iodobenzene diacetate (Bis(acetoxy)iodo benzene; BAIB), the compound (1-2-2) is a compound called Dess-Martin reagent, the compound (2-1-1) is a compound called iodosylbenzene, and the compound (2-2-1) is a compound called 2-iodoxybenzoic acid.

<Method for Producing Hypervalent Iodine Compound>

Since the compound (1) and the compound (2) are known compounds, commercially available products or synthetic products may be used.

The compound (1) and the compound (2) can be produced, for example, using a compound having a basic structure of iodobenzene as a raw material and using a known reaction. More specific details are as follows.

Among the compounds (1), the compound (1-1-2) (BAIB) can be produced by, for example, a production method including a step of reacting iodobenzene, peracetic acid and acetic acid to obtain the compound (1-1-2) (BAIB).

Among the compounds (1), the compound (1-2-2) (Dess-Martin reagent) can be produced by a production method including a step of reacting iodobenzoic acid and potassium peroxymonosulfate to obtain 2-iodoxybenzoic acid and a step of reacting the 2-iodoxybenzoic acid and an acetic anhydride using a catalytic amount of p-toluenesulfonic acid to obtain the compound (1-2-2) (Dess-Martin reagent).

Among the compounds (2), the compound (2-1-1) (iodosylbenzene) can be produced by, for example, a production method including a step of hydrolyzing the compound (1-1-2) (BAIB) to obtain the compound (2-1-1) (iodosylbenzene).

Among the compounds (2), the compound (2-2-1) (2-iodoxybenzoic acid) can be produced by a production method including a step of reacting iodobenzoic acid and potassium peroxymonosulfate to obtain 2-iodoxybenzoic acid.

After the reaction has been completed, each compound may be taken out by carrying out a post-treatment if necessary, according to a known technique. That is, each compound may be taken out by carrying out, if necessary, a post-treatment operation of filtration, washing, extraction, pH adjustment, dehydration, and concentration either alone or in a combination of two or more thereof, and then carrying out concentration, crystallization, reprecipitation, column chromatography or the like. In addition, each taken-out compound may be purified by further carrying out an operation of crystallization, reprecipitation, column chromatography, extraction, and stirring and washing of crystals by a solvent either alone or in a combination of two or more thereof, if necessary, one or more times.

The structure of each compound can be identified by a known technique, for example, nuclear magnetic resonance (NMR) spectroscopy, mass spectrometry (MS), or infrared spectroscopy (IR).

The methods for producing the compound (1) and the compound (2) are not limited to the above-mentioned methods, and the compound (1) and the compound (2) having the desired structure may be produced using other known methods.

<Micelle>

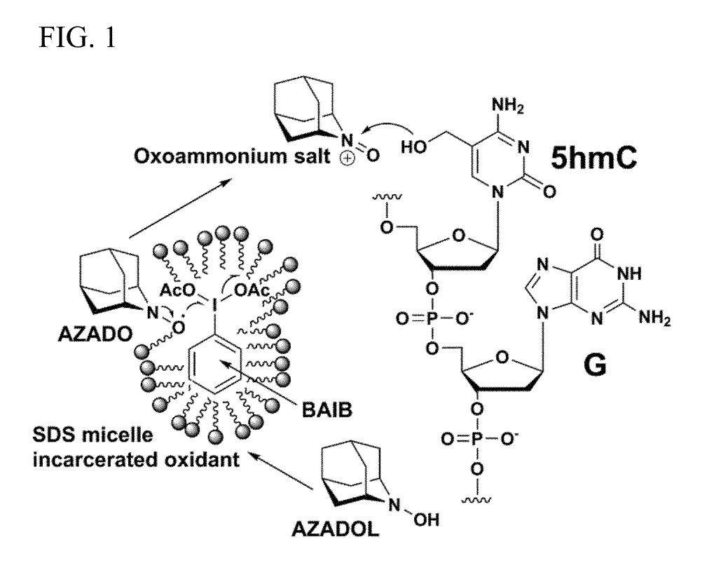

FIG. 1 is a schematic diagram showing an oxidation reaction of hydroxymethylcytosine using an oxidant containing micelle-incarcerated BAIB and AZADOL (registered trademark). As can be seen from FIG. 1, micelle incarceration of the hypervalent iodine compound (compound (1)) such as BAIB by a surfactant results in isolation of the hypervalent iodine compound from a double-stranded polynucleotide that has water-solubility and is a substrate, which thus can inhibit decomposition of the substrate.

In addition, the particle size of the compound (1) or compound (2)-incarcerated micelle in the present embodiment is 50 nm or less, preferably 10 nm or more and 40 nm or less, and more preferably 10 nm or more to 30 nm.

When the particle size of the micelle is more than or equal to the lower limit described above, it is possible to incarcerate the compound (1) or compound (2). When the particle size of the micelle is less than or equal to the upper limit described above, in the case where a double-stranded polynucleotide which is a substrate is of about 300 to 400 bp corresponding to a molecular weight of about 100,000, which results in a large molecular weight difference therebetween, the double-stranded polynucleotide does not enter into the micelle, whereby it is possible to prevent oxidative attack by the compound (1) or the compound (2). In addition, since a molecular weight of a catalyst is about 200 (for example, AZADOL (registered trademark) has a molecular weight of 153), the catalyst is incorporated into the micelle, is reoxidized, and is capable of selectively oxidizing a hydroxy group of hydroxymethylcytosine in a double-stranded polynucleotide which is a substrate.

The particle size of the micelle can be measured by a dynamic light scattering device or a transmission electron microscope.

<<Method for Producing Oxidant>>

In one embodiment, the present invention provides a method for producing a hypervalent iodine compound micelle-incarcerated by a surfactant, including:

(a) a step of mixing the hypervalent iodine compound, acetonitrile, a surfactant, and a solvent, and shaking and stirring the reaction mixture liquid at a temperature of 4° C. or higher and lower than 25° C. until the color of the liquid changes from transparent to pale yellow; and

(b) a step of further shaking and stirring the reaction mixture liquid at a temperature of 25° C. or higher and 30° C. or lower after the step (a),

in which the hypervalent iodine compound has iodobenzene as a basic structure and is hydrophobic.

According to the present embodiment, it is possible to easily produce a hypervalent iodine compound (compound (1) or compound (2)) which has iodobenzene micelle-incarcerated by a surfactant as a basic structure and is hydrophobic.

Hereinafter, the method for producing an oxidant will be described in detail.

First, it is preferred that acetonitrile and a surfactant solution (preferably, acetonitrile:surfactant solution=1:9 in a volume ratio) are mixed in a reaction vessel, and the compound (1) or compound (2) is added thereto. Then, shaking and stirring are carried out at a temperature of preferably 4° C. or higher and lower than 25° C. and more preferably 4° C. or higher and 10° C. or lower until the color of the liquid changes from transparent to pale yellow (step (a)). Since this reaction is an exothermic reaction and acetonitrile having a low boiling point can be prevented from being volatilized if the temperature is lower than 25° C., it is preferred to control the temperature at 4° C. or higher and lower than 25° C. by placing ice water or the like around the reaction vessel.

It is thought that acetonitrile, which is an organic ligand, forms a complex with the compound (1) or compound (2) to thereby contribute to micelle solubilization of the compound (1) or compound (2). The concentration of acetonitrile in the oxidant is preferably 5 to 10%, more preferably 8 to 10%, and particularly preferably 10%. In the case where the concentration of acetonitrile is 10% or less, the micelle formed by a surfactant is not destroyed.

The concentration of the surfactant in the oxidant is preferably 2% or more and 5% or less. In the case where the concentration of the surfactant is 2% or more, it is possible to incarcerate the compound (1) or compound (2) in a micelle, and in the case where the concentration of the surfactant is 5% or less, it is possible to use the surfactant in an amount that is consistent with the cost.

The concentration of the compound (1) or compound (2) in the oxidant is preferably 20 mg/mL or more and 30 mg/mL or less. In the case where the concentration of the compound (1) or compound (2) is 20 mg/mL or more, it is possible to ensure an oxidizing power, and in the case where the concentration of the compound (1) or compound (2) is 30 mg/mL or less, it is possible to obtain an oxidizing power that is consistent with the concentration.

The solvent used in the surfactant solution is not particularly limited as long as it does not inhibit the reaction and it can be used for an oxidation reaction of a water-soluble biopolymer, an example of which is water. Since acetonitrile having a low boiling point is volatilized during the reaction, the surfactant solution may be used in an amount within the range capable of maintaining the concentrations of the compound (1) or compound (2), acetonitrile, and the surfactant at the above-specified concentration, if the amount of the surfactant solution is too small.

The equipment for shaking and stirring is not particularly limited and may be an ultrasonic agitator, an ultrasonic disperser or the like.

The fact that the color of the liquid changes from transparent to pale yellow is an indicator of an oxidative activity of the compound (1) or compound (2) and indicates that compound (1) or compound (2) and acetonitrile form a complex. Therefore, after the color of the liquid changes from transparent to pale yellow, the temperature is increased and the reaction liquid is shaken and stirred at a temperature of 25° C. or higher and 30° C. or lower to completely dissolve the compound (1) or compound (2), thereby accelerating micelle solubilization of the compound (1) or compound (2) (step (b)).

In the present embodiment, the produced oxidant may be stored in a frozen state, but it is preferred that the oxidant is produced immediately prior to use and is completely used on the day of production. This is because the oxidative activity will be lost over time. The index of an oxidative activity is that the color of the liquid exhibits pale yellow, and it can be determined that the oxidative activity is inactivated if the pale yellow color is faded.

<<Oxidation Reagent>>

In one embodiment, the present invention provides an oxidation reagent containing the above-mentioned oxidant, a catalyst which is a compound having an alicyclic heterocyclic ring containing one nitrogen atom as a heteroatom and having a hydroxy group bonded to the nitrogen atom, and a solvent having a pH of 6.0 or more and less than 7.0.

According to the present embodiment, it is possible to obtain a reagent capable of selectively oxidizing a hydroxy group in a water-soluble biopolymer, in an aqueous solvent that reflects an in vivo environment, under mild conditions.

The oxidation reagent of the present embodiment contains the above-mentioned oxidant. A usable surfactant and a method for producing an oxidant are as described above.

<Catalyst>

Further, the oxidation reagent of the present embodiment contains a catalyst which is a compound having an alicyclic heterocyclic ring containing one nitrogen atom as a heteroatom and having a hydroxy group bonded to the nitrogen atom.

In the present embodiment, the “compound having an alicyclic heterocyclic ring containing one nitrogen atom as a heteroatom and having a hydroxy group bonded to the nitrogen atom” which is a catalyst is an N-hydroxy compound represented by the following General Formula (3).

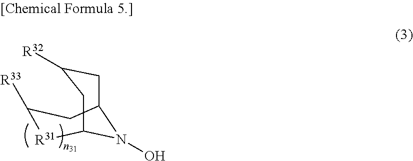

[Group Represented by R³¹]

In General Formula (3), R³¹ has the same definition as in the foregoing section [Groups represented by R¹¹, R¹⁴, R¹⁵, and R¹⁶]. Among them, R³¹ is preferably a hydrogen atom, a halogen atom, a hydroxy group, a C₁₋₃ alkyl group, or a C₁₋₃ alkoxy group.

[Groups Represented by R³² and R³³]

In General Formula (3), R³² and R³³ each independently have the same definition as in the foregoing section [Groups represented by R¹, R¹⁴, R¹⁵, and R¹⁶]. Further, R³² and R³³ may be bonded to each other to form a methylene group.

[n₃₁]

In General Formula (3), n₃₁ represents the number of R³¹ and is an integer of any one of 1 to 8. When n₃₁ is 2 or more, R¹¹'s may be the same or different from one another.

Preferred examples of the compound (3) include a compound represented by the following General Formula (3-1) (hereinafter, sometimes referred to simply as “compound (3-1)”).

These compounds are merely examples of the preferred compound (3), and the preferred compound (3) is not limited thereto.

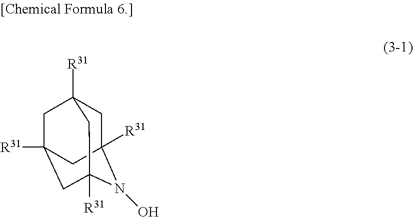

(In the formula, R³¹ is the same as defined above. R³¹'s may be the same or different from one another)

The compound (3-1) is preferably, for example, a compound in which R³¹ is a hydrogen atom, a halogen atom, a hydroxy group, a C₁₋₃ alkyl group, or a C₁₋₃ alkoxy group.

The compound (3-1) is more preferably, for example, a compound in which R³¹ is a hydrogen atom, a fluorine atom, a hydroxy group, a methyl group, or a methoxy group.

Among the compounds (3), preferred examples of the compound (3-1) include N-hydroxy-1-fluoro-2-azaadamantane (1-F-AZADOH), N-hydroxy-5-fluoro-2-azaadamantane (5-F-AZADOH), N-hydroxy-5-fluoro-1-methyl-2-azaadamantane (5-F-1-Me-AZADOH), N-hydroxy-5,7-difluoro-1-methyl-2-azaadamantane (5,7-F₂-1-Me-AZADOH), N-hydroxy-1-methyl-2-azaadamantane (1-Me-AZADOH), N-hydroxy-1-methyl-5-hydroxy-2-azaadamantane (1-Me-5-OH-AZADOH), N-hydroxy-5-methoxy-1-methyl-2-azaadamantane (5-MeO-1-Me-AZADOH), N-hydroxy-5-hydroxy-2-azaadamantane (5-OH-AZADOH), N-hydroxy-9-azabicyclo[3.3.1]nonane (ABNOH), and 2-hydroxy-2-azaadamantane (AZADOL (registered trademark)).

These compounds are merely examples of the preferred compound (3), and the preferred compound (3) is not limited thereto.

Among them, the compound (3) is preferably AZADOL (registered trademark).

[Method for Producing Catalyst]

In the present specification, the compound (3) used as a catalyst can be produced by the methods described in “Y Iwabuchi, Chem. Pharm. Bull. 2013, 61, 1197” and PCT International Publication No. WO 2006/001387. With regard to AZADOL (registered trademark), a commercially available product may be used without synthesis thereof.

[Concentration of Catalyst]

The concentration of the catalyst in the reaction reagent is preferably 1 mg/mL or more and 2 mg/mL or less and more preferably 2 mg/mL.

[Substrate of Catalyst]

In the present embodiment, an oxidation reaction can be carried out in the presence of various compounds having a hydroxy group as a substrate, the compound (3) as a catalyst and the above-mentioned oxidant, under mild conditions.

Further, it is possible to carry out an oxidation reaction even with respect to a compound having a hydroxy group of a sterically hindered structure which has been conventionally difficult to carry out an oxidation reaction with a catalyst such as TEMPO.

In the present embodiment, the alcohol compound to be oxidized is not particularly limited and examples thereof include various compounds having a hydroxy group. As the compound having a hydroxy group, in particular, preferred is a protein or a nucleic acid.

<Solvent>

Further, the oxidation reagent of the present embodiment contains a solvent having a pH of 6.0 or more and less than 7.0. In the case where the solvent has a pH of 6.0 or more and less than 7.0, the oxidation reaction can be carried out smoothly. The solvent preferably has a pH of 6.0 or more and less than 7.0 and more preferably a pH of 6.6 or more and 6.8 or less. The solvent is not particularly limited as long as it is prepared within the above-specified pH range. For example, the solvent may be a phosphate buffer solution. It is preferable that the concentration of the solvent in the reaction reagent is about 0.8 M.

The volume ratio of the solvent having a pH of 6.0 or more and less than 7.0:the micelle-incarcerated compound (1) or compound (2):the catalyst in the reaction reagent is preferably 2:2:1. This is because the oxidative reactivity is lowered if the solvent is increased beyond this ratio.

<<Method for Selectively Oxidizing Hydroxy Group of Target Water-Soluble Biopolymer>>

In one embodiment, the present invention provides a method for selectively oxidizing a hydroxy group of a target water-soluble biopolymer using the above-mentioned oxidation reagent.

According to the present embodiment, it is possible to selectively oxidize a hydroxy group in a water-soluble biopolymer, in an aqueous solvent that reflects an in vivo environment, under mild conditions.

In the present invention and in the present specification, the term “water-soluble biopolymer” refers to a high molecular weight organic compound dissolvable in an aqueous solvent and existing in a living organism, and examples thereof include a protein and a nucleic acid.

FIG. 1 is a schematic diagram showing an oxidation reaction of hydroxymethylcytosine using an oxidant containing micelle-incarcerated BAIB and AZADOL (registered trademark). The method for selectively oxidizing a hydroxy group of a target water-soluble biopolymer using the above-mentioned oxidation reagent will be described in detail below with reference to FIG. 1.

First, a solution containing a target water-soluble biopolymer and the above-mentioned oxidation reagent are mixed and allowed to stand. The oxidation reaction in the present embodiment is characterized by the fact that such an oxidation reaction can be carried out under mild conditions. The reaction can be carried out at a reaction temperature of 4° C. or higher and 40° C. or lower and is preferably carried out at a reaction temperature of 4° C. or higher and 25° C. or lower. In the case where the reaction temperature is 4° C. or higher, it is possible to prevent the precipitation of a salt of a reagent or buffer solution, and in the case where the reaction temperature is 25° C. or lower, it is possible to prevent side reactions such as decomposition of a substrate while maintaining the reaction rate. The reaction time varies depending on the size and amount of the target water-soluble biopolymer, but it is preferably 30 minutes or more and 4 hours or less.

In the mixture of the target water-soluble biopolymer and the oxidation reagent, the micelle-incarcerated BAIB selectively oxidizes a hydroxy group of AZADOL (registered trademark). The oxidized AZADOL (registered trademark) becomes an oxoammonium salt which is an active species through AZADO. This oxoammonium salt selectively oxidizes a hydroxy group of the target water-soluble biopolymer.

Thus, a solution containing a target water-soluble biopolymer and the above-mentioned oxidation reagent are mixed and reacted under mild conditions, whereby it is possible to obtain a target water-soluble biopolymer in which a hydroxy group is oxidized into a formyl group or a ketone group.

<<Kit for Selectively Oxidizing Hydroxy Group of Target Water-Soluble Biopolymer>>

In one embodiment, the present invention provides a kit for selectively oxidizing a hydroxy group of a target water-soluble biopolymer, including a hypervalent iodine compound (compound (1) or compound (2)) which has iodobenzene as a basic structure and is hydrophobic, a surfactant, acetonitrile, a catalyst which is a compound having an alicyclic heterocyclic ring containing one nitrogen atom as a heteroatom and having a hydroxy group bonded to the nitrogen atom, and a solvent having a pH of 6.0 or more and less than 7.0.

According to the kit of the present embodiment, it is possible to selectively oxidize a hydroxy group in a water-soluble biopolymer in an aqueous solvent that reflects an in vivo environment, under mild conditions.

In the present embodiment, the compound (1) or compound (2) and the catalyst are preferably in the form of a powder. As described above, the oxidant containing a micelle-incarcerated compound (1) or compound (2) is preferably produced immediately prior to use.

In addition, since the catalyst has a property of being insoluble in water, acetonitrile and the catalyst are mixed in a volume ratio of 1:1 whereby the catalyst is dissolved in the solvent. The catalyst is prepared at a concentration of 5 mg/mL or more and 10 mg/mL or less, and is mixed such that the final concentration of the catalyst in the reaction solution is 1 mg/mL or more and 2 mg/mL or less.

The surfactant is preferably a liquid with a concentration of about 5% thereof. When producing the micelle-incarcerated compound (1) or compound (2), the surfactant is used to a final concentration of 2% or more and 5% or less.

Acetonitrile is preferably a liquid with a concentration of about 100% thereof. When producing the micelle-incarcerated BAIB, the acetonitrile is used to a final concentration of about 10%.

The solvent having a pH of 6.0 or more and less than 7.0 is preferably a liquid with a concentration of about 2 M thereof. The solvent is used such that the concentration of the solvent in the reaction solution is about 0.8 M.

As described above, the target water-soluble biopolymer may be, for example, a protein or a nucleic acid. In the case of using DNA, it is preferably prepared to achieve a concentration of 5 pM or more and 10 pM or less in the reaction solution.

<<Kit for Detecting Hydroxymethylcytosine in Double-Stranded Polynucleotide>>

In one embodiment, the present invention provides a kit for detecting hydroxymethylcytosine in a double-stranded polynucleotide, including a hypervalent iodine compound (compound (1) or compound (2)) which has iodobenzene as a basic structure and is hydrophobic, a surfactant, acetonitrile, a catalyst which is a compound having an alicyclic heterocyclic ring containing one nitrogen atom as a heteroatom and having a hydroxy group bonded to the nitrogen atom, a solvent having a pH of 6.0 or more and less than 7.0, bisulfite, and a forward and reverse primer set for amplifying a region containing hydroxymethylcytosine in the double-stranded polynucleotide.

According to the kit of the present embodiment, it is possible to easily detect hydroxymethylcytosine in a double-stranded polynucleotide.

In the present embodiment, the compound (1) or compound (2), the surfactant, acetonitrile, the catalyst, and the solvent are as described above.

The term “bisulfite” as used herein refers to a hydrogen sulfite and is for substitution of a cytosine base in a double-stranded polynucleotide to a uracil base.

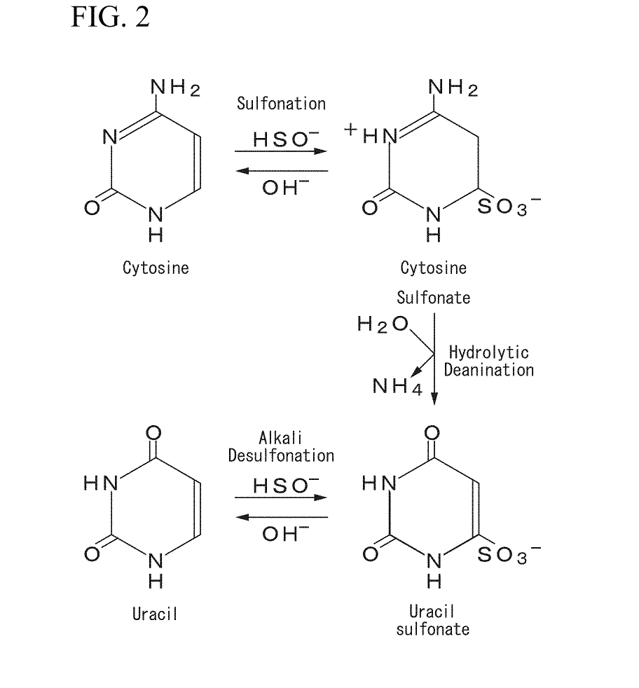

FIG. 2 is a diagram showing a reaction process of converting a cytosine base into a uracil base by bisulfite. Further, it is known also for hydroxymethylcytosine that a hydroxy group is oxidized into a formyl group which is thereby then converted into uracil by bisulfite treatment.

The kit of the present embodiment includes a forward and reverse primer set for amplifying a region containing hydroxymethylcytosine in a double-stranded polynucleotide. The base sequence of the primer set may be appropriately designed depending on the region containing hydroxymethylcytosine in a target double-stranded polynucleotide.

The kit of the present embodiment may include at least any one of one or a plurality of control cells and nucleic acids. For example, the control nucleic acid encompasses a nucleic acid including a gene sequence that is either accessible in essentially all cells of an animal (for example, a housekeeping gene sequence or a promoter thereof), or inaccessible in essentially all cells of an animal.

The kit of the present embodiment may include one or a plurality of sets of primers for amplifying such a gene sequence (regardless of whether or not a gene sequence or a cell is actually included in the kit).

The kit of the present embodiment may include at least any one of a DNA modifying agent, a cell permeabilizing agent and a cell disintegrating agent, and one or a plurality of primer sets for amplifying a control DNA region.

For example, in the case where the DNA modifying agent is a restriction enzyme, the kit of the present embodiment may include at least two primer sets consisting of a first primer set for amplifying a portion of a DNA region containing at least one (for example, one, two, three or four) potential cleavage site of a restriction enzyme per one DNA region (for example, which is useful for calculating the number of unmodified copies), and at least a second primer set for amplifying a portion of a DNA region not containing a potential cleavage site (for example, which is useful for calculating the total number of copies).

The kit of the present embodiment may include one or a plurality of the following:

(i) a permeabilizing agent or disintegrating agent of a cell membrane;

(ii) a restriction enzyme, a DNase, or other DNA modifying agents;

(iii) a “stop” solution that can prevent further modification by a modifying agent;

(iv) a material for carrying out at least any one of extraction and purification of a nucleic acid (for example, a spin column for carrying out at least any one of purification of genomic DNA and removal of non-DNA components such as components of the “stop” solution); and

(vi) a reagent for PCR or qPCR amplification of DNA (and optionally, a single mixture containing all the components required for PCR or qPCR, in addition to at least any one of a template and a polymerase).

<<Method for Detecting Hydroxymethylcytosine in Double-Stranded Polynucleotide>>

In one embodiment, the present invention provides a method for detecting hydroxymethylcytosine in a double-stranded polynucleotide, including:

(A) a step of mixing a sample containing a double-stranded polynucleotide having hydroxymethylcytosine, the above-mentioned oxidant, a catalyst which is a compound having an alicyclic heterocyclic ring containing one nitrogen atom as a heteroatom and having a hydroxy group bonded to the nitrogen atom, and a solvent having a pH of 6.0 or more and less than 7.0 and oxidizing hydroxymethylcytosine to convert into formylcytosine;

(B) a step of treating a sample containing a double-stranded polynucleotide having the converted formylcytosine with bisulfite to convert formylcytosine into uracil;

(C) a step of adding forward and reverse primers for amplifying a region containing hydroxymethylcytosine in the double-stranded polynucleotide to a double-stranded polynucleotide having the converted uracil to amplify the polynucleotide; and

(D) a step of comparing with a sample containing a double-stranded polynucleotide subjected to steps (A) to (C) without adding an oxidant as a control to detect hydroxymethylcytosine in a double-stranded polynucleotide.

According to the present embodiment, it is possible to easily detect hydroxymethylcytosine in a double-stranded polynucleotide.

Details of the detection method of the present embodiment will be described below.

First, a sample containing a double-stranded polynucleotide having hydroxymethylcytosine, an oxidant containing micelle-incarcerated BAIB, a catalyst, and a solvent having a pH of 6.0 or more and less than 7.0 are mixed (step A). As in the case of the foregoing section <Oxidation reagent>, the volume ratio of the solvent having a pH of 6.0 or more and less than 7.0:the micelle-incarcerated compound (1) or compound (2):the catalyst is preferably 2:2:1.

As in the case of the foregoing section <Method for selectively oxidizing hydroxy group of target water-soluble biopolymer>, the reaction is preferably carried out at room temperature. The reaction time varies depending on the size and amount of the target water-soluble biopolymer, but it is preferably 30 minutes or more and 4 hours or less.

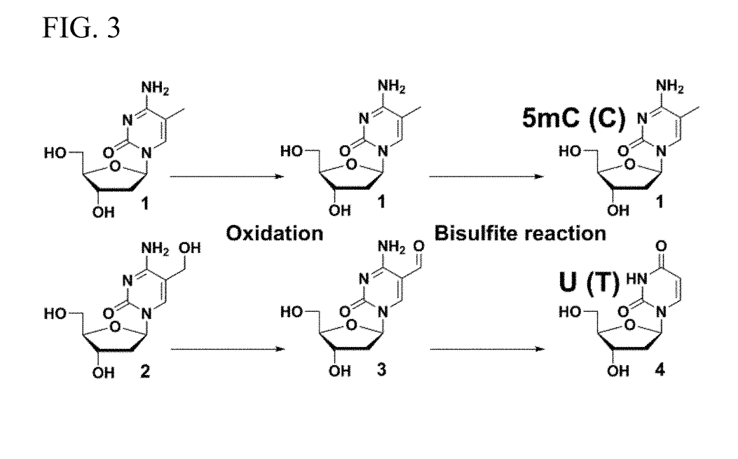

FIG. 3 is a diagram comparing changes in hydroxymethylcytosine and methylcytosine in a double-stranded polynucleotide before and after subjecting to an oxidation reaction and a bisulfite treatment.

In the reaction solution, hydroxymethylcytosine (2) in a double-stranded polynucleotide is oxidized and converted into formylcytosine (3) (step A). On the other hand, methylcytosine (1) remains as methylcytosine without being changed, even after subjecting to an oxidation reaction and a bisulfite treatment.

It is preferable to carry out a step of removing the oxidant after conversion into formylcytosine (3). Specifically, there may be mentioned a method of extracting a double-stranded polynucleotide by an organic solvent or the like and then purifying the extract by using a spin column or the like.

Next, the reaction solution is subjected to a bisulfite treatment. The bisulfite treatment is preferably carried out under the reaction conditions of a temperature of 80° C. or higher and lower than 100° C. for 30 minutes or more and 1 hour or less. In the reaction solution, formylcytosine (3) in a double-stranded polynucleotide is sulfonated and is further converted into sulfonated uracil by a hydrolytic deamination reaction. Subsequently, an alkali treatment is followed to remove the sulfone group from the sulfonated uracil which is then converted into uracil (4) (step B).

Subsequently, forward and reverse primers for amplifying a region containing hydroxymethylcytosine in the double-stranded polynucleotide are added to the double-stranded polynucleotide subjected to a bisulfite treatment, followed by amplification via PCR, qPCR, or the like (step C).

Those skilled in the art can determine the PCR or qPCR conditions depending on the length and sequence characteristics of the double-stranded polynucleotide, according to a known method.

Subsequently, a sample containing the double-stranded polynucleotide subjected to steps (A) to (C) without adding an oxidant as a control is subjected to base sequence comparison by a Sanger sequencing method (step D). By comparison with the control, it is possible to detect hydroxymethylcytosine in a double-stranded polynucleotide.

Hydroxymethylcytosine (5hmC) is thought to be involved in switching on or off of gene expression and its abundance differs greatly depending on the type of cell. Further, it is known that the largest amount of 5hmC is present in the central nervous system. Further, it is known that the content of 5hmC in the mouse hippocampus and cerebellum increases with aging. Therefore, the detection of 5hmC attracts attention in the field of epigenetics.

The term “epigenetics” as used herein refers to a system that controls and communicates gene expression independent of DNA sequence changes and its academic field, and is a mechanism independent of changes in the DNA base sequence (mutations) while having the genetic characteristics that are inherited to daughter cells through cell division. Such a control changes dynamically due to an environmental factor such as diet, air pollution, smoking, or exposure to oxidative stress, while it is a chemically stable modification. That is, epigenetics is considered to be a mechanism that will be a bridge between genes and environmental factors.

The detection of 5hmC is therefore expected to make a great contribution to the study on the presence or absence of the onset of disease and difference in the degree of symptoms thereof, or stem cell and regenerative medicine research.

The embodiment of the present invention has been described in detail with reference to the accompanying drawings, but the specific configuration is not limited to such an embodiment and also includes configurations within the scope not deviating from the gist of the present invention.

### EXAMPLES

Hereinafter, the present invention will be described in further detail with reference to the following Examples, but the present invention is not limited thereto.

### Example 1

1-1. Preparation of Oxidant

Powdered BAIB (manufactured by Wako Pure Chemical Industries, Ltd.) (20 mg) was added to a mixed solution of a 5% SDS aqueous solution (0.9 mL) and 100% acetonitrile (0.1 mL) which was then shaken and stirred for about 10 minutes in an ultrasonic agitator where ice was suspended. When the liquid showed a pale yellow color, the ice in the ultrasonic agitator was removed and the mixture liquid was further shaken and stirred at room temperature for complete dissolution, thereby obtaining an oxidant containing micelle-incarcerated BAIB. The oxidant contained 4.5% SDS, 10% acetonitrile, and 20 mg/mL BAIB.

Further, when the BAIB-incarcerated micelle was measured using a dynamic light scattering device, the particle size thereof was about 18 nm, and when the BAIB-incarcerated micelle was measured using a transmission electron microscope, the particle size thereof was about 26 nm.

1-2. Preparation of Catalyst

Powdered AZADOL (manufactured by Wako Pure Chemical Industries, Ltd.) (5 mg) was added to a mixed solution of 100% acetonitrile (0.25 mL) and water (0.25 mL) which was then shaken and stirred for about 10 minutes in an ultrasonic agitator to obtain a catalyst solution. The catalyst solution contained 50% acetonitrile and 10 mg/mL AZADOL.

1-3. Oxidation Reaction of Hydroxylmethylcytosine in Substrate DNA

The base sequences of 10-bp double-stranded DNAs (manufactured by Gene Design, Inc.) used as a substrate are shown in Table 1. Each of the substrate DNAs was diluted in a buffer solution (10 mM Tris-HCl (pH 8.0), 50 mM NaCl, and 1 mM EDTA).

TABLE 1

| | | | SEQ ID |
|---|---|---|---|
| Substrate DNA | | Base sequence | NO: |
| (i) | Sense strand | 5′-CAATC5hmCGGTA-3′ | 1 |
| | Antisense | 5′-GTTAGGCCAT-3′ | 2 |
| | strand | | |
| (ii) | Sense strand | 5′-CAATCCGGTA-3′ | 3 |
| | Antisense | 5′-GTTAGGCCAT-3′ | 2 |
| | strand | | |
| (iii) | Sense strand | 5′-CAATC5mCGGTA-3′ | 4 |
| | Antisense | 5′-GTTAGGCCAT-3′ | 2 |
| | strand | | |

A sample solution (1 μL) in which each substrate DNA (0.6 nmol) was dissolved, a 2 M phosphate buffer solution (pH 6.8) (24 μL), 20 mg/mL BAIB (24 μL) and 10 mg/mL AZADOL (12 μL) were added and mixed in a PCR tube. The mixture was allowed to stand for 30 minutes in a bath which had been kept at a low temperature by ice, thereby carrying out an oxidation reaction. Aniline (120 μL) was added to stop the oxidation reaction, followed by vortexing. After carrying out centrifugation on a table top centrifuge, a step of removing the upper brown organic layer and adding toluene (120 μL) to remove the residual oxidant was repeated three times. In addition, a control with no addition of an oxidant was also prepared.

1-4. Bisulfite Treatment

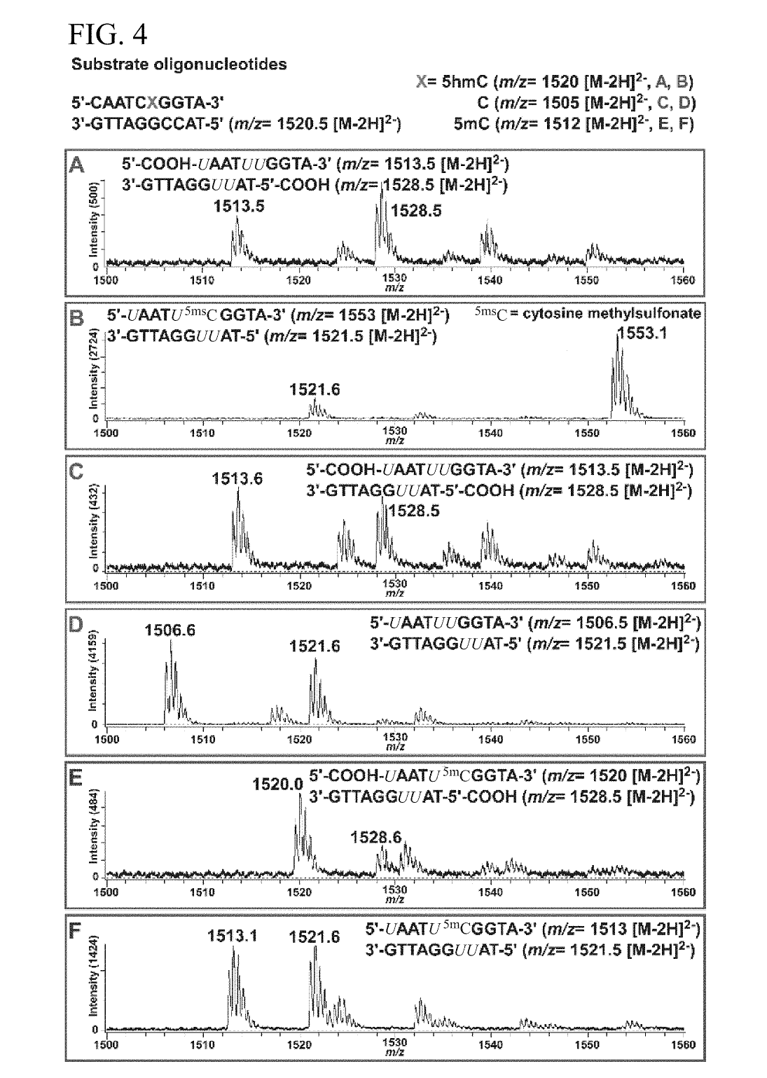

10 M bisulfite (120 μL) was added to the solution containing the extracted substrate DNA which was then maintained at 90° C. for 1 hour. Subsequently, DNA was purified by ODS-HPLC. 0.2 M NaOH (10 μL) was added thereto, followed by standing at 37° C. for 30 minutes to carry out a desulfonation treatment. The resulting substrate DNA-containing solutions were analyzed using LC-ESI-MS (JMS-T100LP manufactured by JEOL Ltd.). The results are shown in FIG. 4.

In FIG. 4, A, C and E are the results of test samples subjected to an oxidation reaction, in which the 5′ terminal was oxidized to convert into a carboxyl group. B, D and F are the results of control samples not subjected to an oxidation reaction.

When comparing A and B in FIG. 4, hydroxymethylcytosine located at the 6^{th} position counting from the 5′ terminal was converted into uracil in the test sample subjected to an oxidation reaction, whereas the corresponding residue was cytosine methylenesulfonate and was not converted into uracil in the control.

In addition, when comparing A and C in FIG. 4, peak values of the mass spectra were the same therebetween.

In addition, as can be seen from E and F in FIG. 4, an effect of an oxidation reaction on methylcytosine was not observed.

From the foregoing, it was confirmed that hydroxymethylcytosine is oxidized by using micelle-incarcerated BAIB and AZADOL. It was found that the position of hydroxymethylcytosine can be identified by comparing the case subjected to an oxidation reaction and the case not subjected to an oxidation reaction.

### Example 2

2-1. Preparation of Oxidant

The oxidant was prepared in the same manner as in 1-1 of Example 1.

2-2. Preparation of Catalyst

The catalyst was prepared in the same manner as in 1-2 of Example 1.

2-3. Oxidation Reaction of Hydroxylmethylcytosine in Substrate DNA

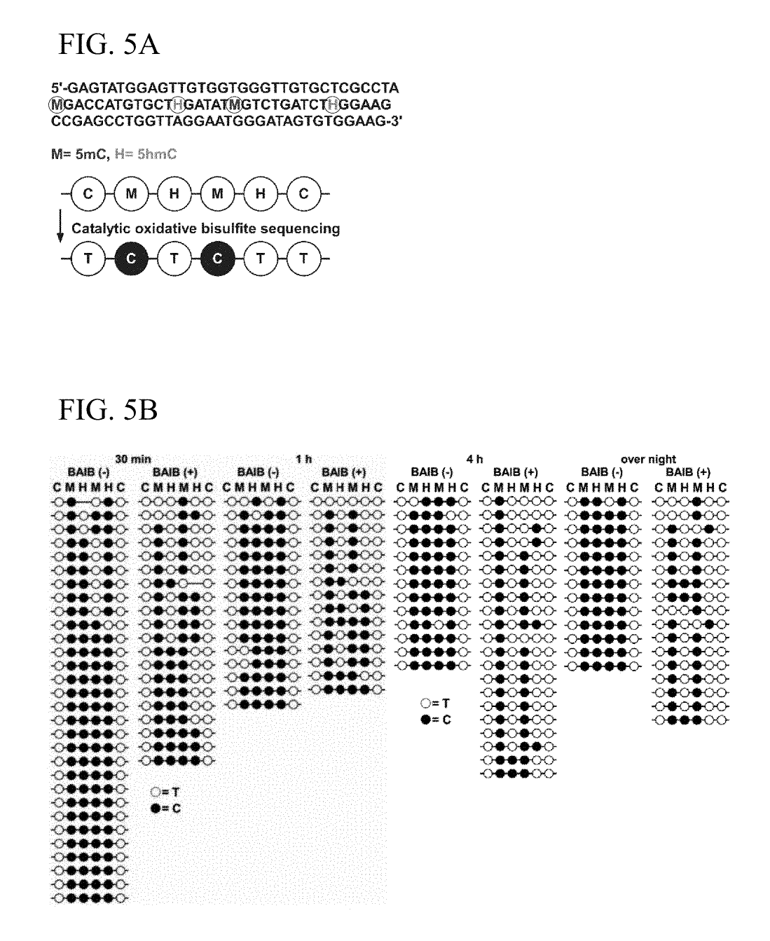

100-bp double-stranded DNA (manufactured by Gene Design, Inc.) was used as a substrate. In the base sequence, a sense strand was set forth in SEQ ID NO: 5 and an antisense strand was set forth in SEQ ID NO: 6. FIG. 5A is a schematic diagram showing the position of hydroxymethylcytosine and methylcytosine in 100-bp double-stranded DNA which is a substrate, and the conversion of cytosine and hydroxymethylcytosine into uracil in substrate DNA by means of an oxidation reaction and a bisulfite treatment.

A sample solution (0.5 μL) in which substrate DNA (0.1 nmol) was dissolved, a 2 M phosphate buffer solution (pH 6.8) (20 μL), 20 mg/mL BAIB (20 μL) and 10 mg/mL AZADOL (10 μL) were added and mixed in a PCR tube. The mixture was subjected to an oxidation reaction for 30 minutes, 1 hour, 4 hours and 12 hours in a bath which had been kept at a low temperature by ice. Aniline (100 μL) was added to stop the oxidation reaction, followed by vortexing. After carrying out centrifugation on a table top centrifuge, a step of removing the upper brown organic layer and adding toluene (100 μL) to extract the substrate DNA was repeated three times. In addition, a control not subjected to an oxidation reaction was also prepared.

2-4. Bisulfite Treatment

10 M bisulfite (100 μL) was added to the solution containing the extracted substrate DNA which was then maintained at 90° C. for 1 hour. Subsequently, DNA was purified using an EZ DNA Methylation kit (manufactured by Zymo Research Corporation).

2-5. Amplification of Substrate DNA

The purified substrate DNA was amplified by PCR. Taq polymerase is used as an enzyme, and base sequences of primers are shown in Table 2.

TABLE 2

| | | SEQ ID |
|---|---|---|
| | Base sequence | NO: |
| Forward primer | 5′-GAGTATGGAGTTGTGGTGGG-3′ | 7 |
| Reverse primer | 5′-GATAGTGTGGAAG-3′ | 8 |

The amplified PCR product was purified using a PCR purification kit.

2-6. Detection of Hydroxylmethylcytosine by Sanger Sequencing Method

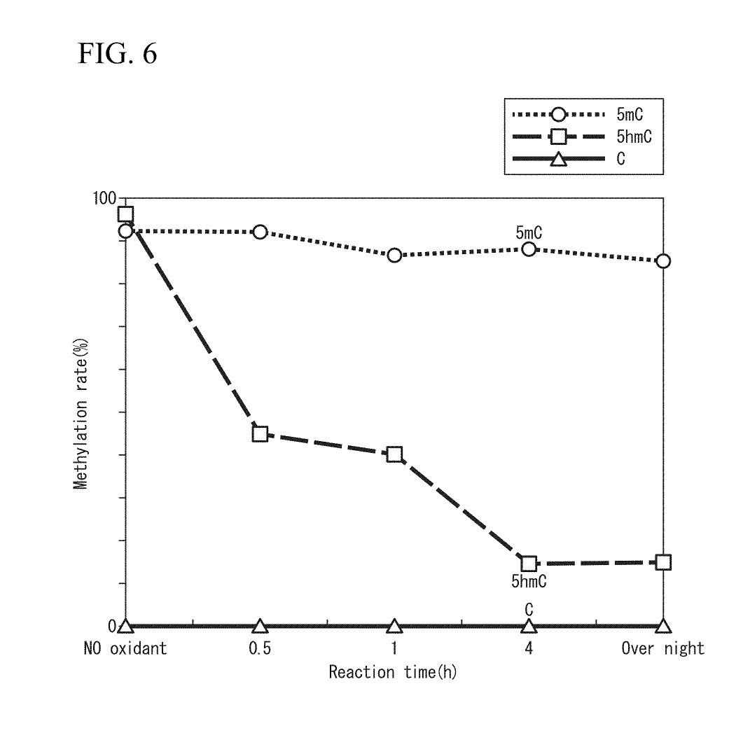

The purified PCR product was inserted into a vector, and the base sequence thereof was identified by a Sanger sequencing method. The results are shown in FIG. 5B and FIG. 6.

As can be seen from FIG. 5B and FIG. 6, it was found that the percentage of hydroxymethylcytosine converted into uracil was proportional to the oxidation reaction time. Further, with respect to 100-bp substrate DNA containing two hydroxymethylcytosines, it was found that about 86% of hydroxymethylcytosines in the substrate DNA was converted into uracil, by carrying out an oxidation reaction for 4 hours or more.

From the foregoing results, it has been found that it is possible to selectively oxidize a hydroxy group in a water-soluble biopolymer, in an aqueous solvent that reflects an in vivo environment, under mild conditions. Further, it has been found that it is possible to easily detect hydroxymethylcytosine in a double-stranded polynucleotide.

### INDUSTRIAL APPLICABILITY

According to the present invention, it is possible to selectively oxidize a hydroxy group in a water-soluble biopolymer, in an aqueous solvent that reflects an in vivo environment, under mild conditions. Further, according to the present invention, it is possible to easily detect hydroxymethylcytosine in a double-stranded polynucleotide.

### REFERENCE SIGNS LIST

1 . . . methylcytosine (5mC), 2 . . . hydroxymethylcytosine (5hmC), 3 . . . formylcytosine (5fC), 4 . . . uracil

1. An oxidant comprising a hypervalent iodine compound which has iodobenzene micelle-incarcerated by a surfactant as a basic structure and is hydrophobic.

2. The oxidant according to claim 1, wherein the surfactant is an anionic surfactant.

3. The oxidant according to claim 1, wherein the hypervalent iodine compound is an iodobenzene diacetate, a Dess-Martin reagent or a 2-iodoxybenzoic acid.

4. The oxidant according to claim 1, wherein the particle size of the micelle is 50 nm or less.

5. (canceled)

6. (canceled)

7. An oxidation reagent, comprising:
the oxidant according to claim 1;
a catalyst which is a compound having an alicyclic heterocyclic ring containing one nitrogen atom as a heteroatom and having a hydroxy group bonded to the nitrogen atom; and
a solvent having a pH of 6.0 or more and less than 7.0.

8. A method for selectively oxidizing a hydroxy group of a target water-soluble biopolymer using the oxidation reagent according to claim 7.

9. A kit comprising:
a hypervalent iodine compound which has iodobenzene as a basic structure and is hydrophobic;
a surfactant;
acetonitrile;
a catalyst which is a compound having an alicyclic heterocyclic ring containing one nitrogen atom as a heteroatom and having a hydroxy group bonded to the nitrogen atom; and
a solvent having a pH of 6.0 or more and less than 7.0.

10. The kit according to claim 9, further comprising:
bisulfite; and
a forward and reverse primer set for amplifying a region containing hydroxymethylcytosine in the double-stranded polynucleotide.

11-18. (canceled)


SEQUENCE LISTING (8 sequences)

SEQ ID NO: 1 - DNA, 10 bp, Artificial Sequence
  Synthesized
  modified_base (6)..(6): n indicates 5-hydroxymethylcytosine.
  misc_feature (6)..(6): n is a, c, g, or t
caatcnggta

SEQ ID NO: 2 - DNA, 10 bp, Artificial Sequence
  Synthesized
gttaggccat

SEQ ID NO: 3 - DNA, 10 bp, Artificial Sequence
  Synthesized
caatccggta

SEQ ID NO: 4 - DNA, 10 bp, Artificial Sequence
  Synthesized
  modified_base (6)..(6): n indicates 5-methylcytosine.
  misc_feature (6)..(6): n is a, c, g, or t
caatcnggta

SEQ ID NO: 5 - DNA, 100 bp, Artificial Sequence
  Synthesized
  modified_base (34)..(34): n indicates 5-methylcytosine.
  misc_feature (34)..(34): n is a, c, g, or t
  modified_base (46)..(46): n indicates 5-hydroxymethylcytosine.
  misc_feature (46)..(46): n is a, c, g, or t
  modified_base (52)..(52): n indicates 5-methylcytosine.
  misc_feature (52)..(52): n is a, c, g, or t
  modified_base (62)..(62): n indicates 5-hydroxymethylcytosine.
  misc_feature (62)..(62): n is a, c, g, or t
gagtatggagttgtggtgggttgtgctcgcctangaccatgtgctngatatngtctgatctnggaagccgagcctggttaggaatgggatagtgtggaag

SEQ ID NO: 6 - DNA, 100 bp, Artificial Sequence
  Synthesized
cttccacactatcccattcctaaccaggctcggcttccgagatcagacgatatcgagcacatggtcgtaggcgagcacaacccaccacaactccatactc

SEQ ID NO: 7 - DNA, 20 bp, Artificial Sequence
  Synthesized
gagtatggagttgtggtggg

SEQ ID NO: 8 - DNA, 13 bp, Artificial Sequence
  Synthesized
gatagtgtggaag